(19) United States
(12) Patent Application Publication
(10) Pub. No.: US 2007/0156209 A1
(43) Pub. Date: Jul. 5, 2007

(54) System for the treatment of heart tissue

(75) Inventors: Michael D. Laufer, Menlo Park, CA (US); Steven H. Trebotich, Newark, CA (US); Jerome Jackson, Los Altos, CA (US)

Correspondence Address: THELEN REID BROWN RAYSMAN & STEINER LLP, P. O. BOX 640640, SAN JOSE, CA, 95164-0640, US

(73) Assignee: Co-Repair, Inc., Sunnyvale, CA (US)

(21) Appl. No.: 11/599,166

(22) Filed: Nov. 13, 2006

Related U.S. Application Data
(63) Continuation-in-part of application No. 11/035,657, filed on Jan. 14, 2005.

(51) Int. Cl. A61F 7/00; A61F 7/12

(52) U.S. Cl. 607/96; 607/113

(57) ABSTRACT

A system for treating an affected portion in a heart comprises a catheter having a first end and a second end; a mono-polar or bi-polar electrode coupled to the first end, wherein the electrode is adapted to be inserted into heart tissue; a power source coupled to the second end and configured to energize the electrode, wherein the electrode emits a radio frequency (RF) signal upon being energized to heat the affected portion to a desired temperature; a temperature feedback control coupled to the electrode and the power source, wherein electrode is configured to alter the emitted RF signal based on a measured temperature of the affected portion. A rotatable member is configured to allow a first portion of the catheter to freely rotate with respect to a second portion of the catheter.

### STATEMENT OF RELATED APPLICATION

The present application is a continuation-in-part of co-pending U.S. patent application Ser. No. 11/035,657 filed Jan. 14, 2005, in the name of inventor Michael D. Laufer.

### TECHNICAL FIELD

The subject matter discussed herein is directed the treatment of heart tissue.

### BACKGROUND

As is well known, the heart has four chambers for receiving and pumping blood to various parts of the body. During normal operation of the heart, oxygen-poor blood returning from the body enters the right atrium. The right atrium fills with blood and eventually contracts to expel the blood through the tricuspid valve to the right ventricle. Contraction of the right ventricle ejects the blood in a pulse-like manner into the pulmonary artery and each lung. The oxygenated blood leaves the lungs through the pulmonary veins and fills the left atrium. The left atrium fills with blood and eventually contracts to expel the blood through the mitral valve to the left ventricle. Contraction of the left ventricle forces blood through the aorta to eventually deliver the oxygenated blood to the rest of the body.

Myocardial infarction (i.e., heart attack) can result in congestive heart failure. Congestive heart failure is a condition wherein the heart can not pump enough blood. When patients have a heart attack, part of the circulation to the heart wall muscle is lost usually due to a blood clot which dislodges from a larger artery and obstructs a coronary artery. If the clot is not dissolved within about 3 to 4 hours, the muscle which lost its blood supply necroses and subsequently becomes a scar. The scarred muscle is not contractile, and therefore it does not contribute to the pumping ability of the heart. In addition, the scarred muscle is elastic (i.e., floppy) which further reduces the efficiency of the heart because a portion of the force created by the remaining healthy muscle bulges out the scarred tissue (i.e., ventricular aneurism) instead of pumping the blood out of the heart.

Congestive heart failure is generally treated with lots of rest, a low-salt diet, and medications such as A.C.E. inhibitors, digitalis, vasodilators and diuretics. In some myocardial infarction instances, the scarred muscle is cut out of the heart and the remaining portions of the heart are sutured (i.e., aneurismechtomy). In limited circumstances a heart transplant may be performed. The condition is always progressive and eventually results in patient death.

Collagen-containing tissue is ubiquitous in normal human body tissues. Collagen makes up a substantial portion of scar tissue, including cardiac scar tissue resulting from healing after a heart attack. Collagen demonstrates several unique characteristics not found in other tissues. Intermolecular cross links provide collagen-containing tissue with unique physical properties of high tensile strength and substantial elasticity. A property of collagen is that collagen fibers shorten when heated. This molecular response to temperature elevation is believed to be the result of rupture of the collagen stabilizing cross links and immediate contraction of the collagen fibers to about one-third of their original length. If heated to approximately 70 degrees Centigrade, the cross links will again form at the new dimension. If the collagen is heated above about 85 degrees Centigrade, the fibers will still shorten, but crosslinking will not occur, resulting in denaturation. The denatured collagen is quite expansile and relatively inelastic. In living tissue, denatured collagen is replaced by fibroblasts with organized fibers of collagen than can again be treated if necessary. Another property of collagen is that the caliber of the individual fibers increases greatly, over four fold, without changing the structural integrity of the connective tissue.

### OVERVIEW

In an embodiment, a system and method for treating an affected portion of heart tissue including, but not limited to, inserting a mono-polar or bi-polar electrode into heart tissue at least proximal to the affected portion; energizing the electrode to emit a radio frequency (RF) signal to heat the affected portion; and measuring a temperature of the affected portion, wherein the energizing of the electrode is associated with the measured temperature. In an embodiment, the electrode is no longer energized upon the measured temperature reaching a desired temperature. In an embodiment, the method further comprises transmitting a signal associated with the measured temperature to a processor, wherein the processor compares the measured temperature to a designated termination temperature. In an embodiment, power supplied to energize the electrode is altered based on the transmitted signal. In an embodiment, inserting further comprises rotating the electrode about an axis into the heart tissue, wherein the electrode includes a helical configuration. The electrode may be inserted directly into the affected portion or inserted directly into healthy tissue to treat the affected portion in at least one of below the healthy tissue or adjacent to the healthy tissue. In an embodiment, the desired temperature is in the range of about 40 degrees Celsius to about 75 degrees Celsius.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

In the drawings:

FIG. 1A illustrates an overall schematic diagram of the tissue repair device in accordance with an embodiment.

FIGS. 1B-1C illustrate views of the tissue insertion component of the tissue repair device in accordance with an embodiment.

FIGS. 2A-2C illustrate views of the rotatable coupling of the tissue repair device in accordance with an embodiment.

FIG. 3 is a view of a device for the treatment of infarcted heart tissue in accordance with an embodiment.

FIG. 4 is a view of the device shown in FIG. 3 taken along line 4-4 in accordance with an embodiment.

FIG. 5 is a view a portion of the device within a catheter in accordance with an embodiment.

FIG. 6 is view of the device within a heart in accordance with an embodiment.

FIG. 7 is view of the device in contact with a heart wall in accordance with an embodiment.

FIG. 8A is a view of a device within a heart in accordance with an embodiment.

FIG. 8B is view of the device shown in FIG. 8A taken along line 8-8 in accordance with an embodiment.

FIG. 9 is a view of a device for the treatment of infarcted heart tissue in accordance with an embodiment.

FIG. 10 is a view of the embodiment of FIG. 9 without the protective material in accordance with an embodiment.

FIG. 11 is a view of the device in FIG. 10 within a heart in accordance with an embodiment.

FIG. 12 is a view of a device for the treatment of infarcted heart tissue in accordance with an embodiment.

FIG. 13 is a flow chart illustrating the method of utilizing the tissue repair device of one or more embodiments.

### DESCRIPTION OF EXAMPLE EMBODIMENTS

Example embodiments are described herein in the context of a system and method to heal an infarct tissue. Those of ordinary skill in the art will realize that the following description is illustrative only and is not intended to be in any way limiting. Other embodiments will readily suggest themselves to such skilled persons having the benefit of this disclosure. Reference will now be made in detail to implementations of the example embodiments as illustrated in the accompanying drawings. The same reference indicators will be used throughout the drawings and the following description to refer to the same or like items.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

In accordance with this disclosure, the components, process steps, and/or data structures described herein may be implemented using various types of operating systems, computing platforms, computer programs, and/or general purpose machines. In addition, those of ordinary skill in the art will recognize that devices of a less general purpose nature, such as hardwired devices, field programmable gate arrays (FPGAs), application specific integrated circuits (ASICs), or the like, may also be used without departing from the scope and spirit of the inventive concepts disclosed herein. Where a method comprising a series of process steps is implemented by a computer or a machine and those process steps can be stored as a series of instructions readable by the machine, they may be stored on a tangible medium such as a computer memory device (e.g., ROM (Read Only Memory), PROM (Programmable Read Only Memory), EEPROM (Electrically Eraseable Programmable Read Only Memory), FLASH Memory, Jump Drive, and the like), magnetic storage medium (e.g., tape, magnetic disk drive, and the like), optical storage medium (e.g., CD-ROM, DVD-ROM, paper card, paper tape and the like) and other types of program memory.

In general, a power generating device provides modulated power to a helical shaped electrode which emits RF signals at a selected frequency and magnitude when energized. The RF signals emitted from the electrode are converted into heat by the affected tissue, whereby heating of the affected tissue to a desired temperature causes reduction of the surface area in the affected infarct tissue without ablating the affected tissue or damaging the healthy tissue surrounding the affected area.

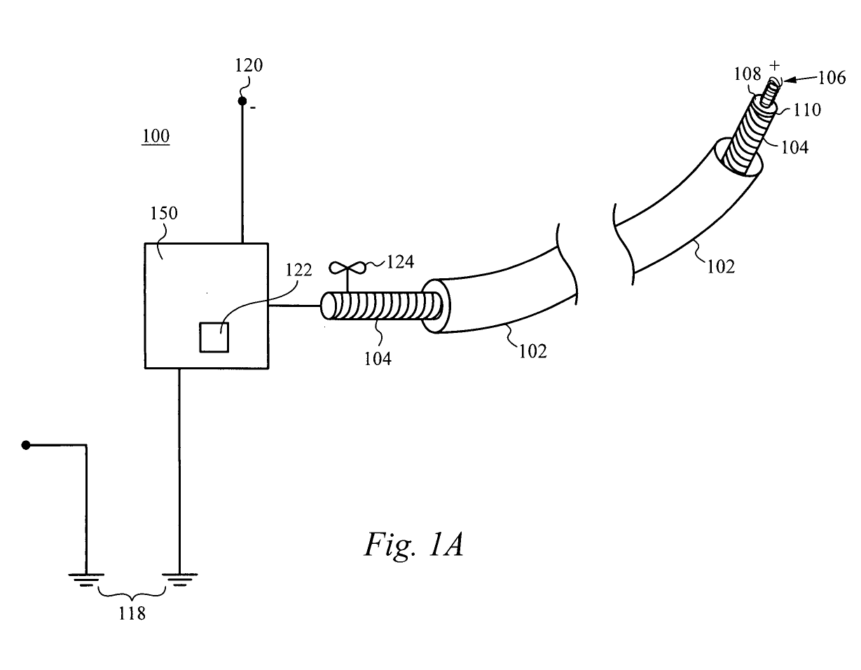

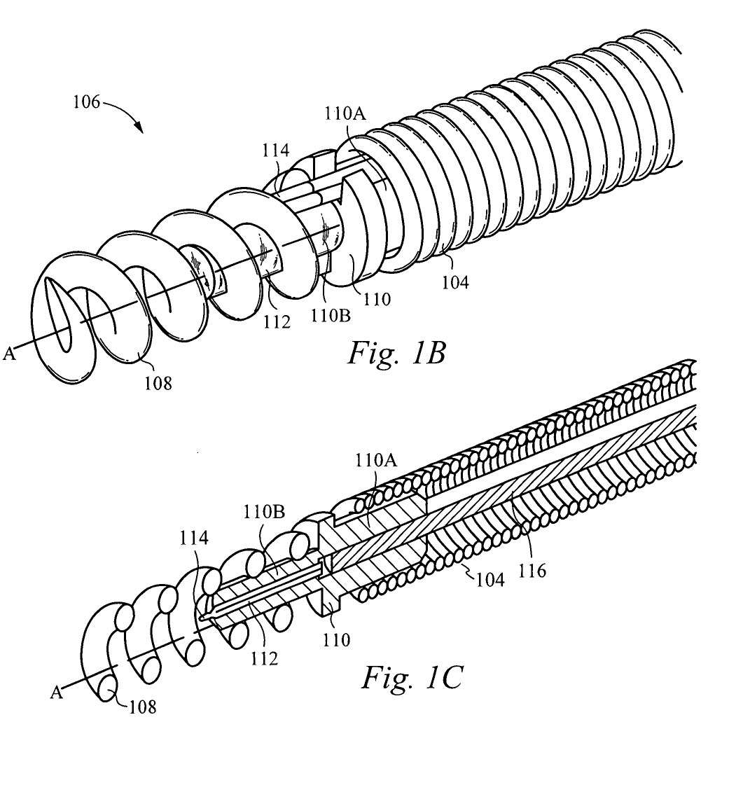

FIG. 1A illustrates an overall schematic diagram of the tissue repair device 100 in accordance with an embodiment. In an embodiment, the tissue repair device 100 includes a catheter sleeve 102 configured to receive a flexible cable catheter 104 of the tissue repair device 100. As shown in FIG. 1A, a tissue insertion device 106 is attached to a distal end of the flexible cable 104. The flexible cable 104 is rotatable and is configured to transmit a torque to the tissue insertion device 106 when rotated at any location along the length of the cable 104. The flexible cable 104 is removably insertable into the lumen of the catheter sleeve 102 so that the cable 104 is able to slide therein to the targeted infarct area of the heart tissue after the catheter sleeve 102 is inserted into the patient.

As shown in FIG. 1A, an end of the flexible cable 104 is shown schematically proximal to a power supply device 150 outside of the patient's body which generates radio-frequency (RF) signals. In addition, the tissue insertion device 106 at the opposite end of the flexible cable 104 includes a RF electrode 108 extending from a coupling connector 110. The details of the tissue insertion device 106 will now be described.

FIGS. 1B and 1C illustrate detailed views of the tissue insertion portion 106 in accordance with an embodiment. As shown in FIGS. 1B and 1C, the tissue insertion device 106 includes the coupling connector 110, a thermocouple sensor 112 and a corkscrew-shaped RF electrode 108. In particular to the embodiment shown in FIGS. 1B and 1C, the coupling connector 110 has an inner portion 110A having a diameter configured to allow the inner portion 110A to fit within the inside of the flexible cable 104. As shown in an embodiment in FIG. 1C, the coupling connector 110 is coupled to a rotational stability wire 116 which extends to the distal end of the flexible cable 104 and is fixed with respect to the flexible cable 104. In the embodiment shown in FIGS. 1B and 1C, the coupling connector 110 includes an outer portion 110B which extends outside of the flexible cable 104 and is configured to be substantially surrounded by the corkscrew shaped electrode 108 in an embodiment shown in FIGS. 1B and 1C. In an embodiment, the coupling connector 110 is made of Lexan, Nylon or any other appropriate rigid material which is non-conductive.

In an embodiment, the coupling connector 110 includes an inner shaft 114 which houses a portion of the thermocouple sensor 112. An aperture at the end in the coupling connector 110 may be formed in communication with the inner shaft 114 to allow a portion of the thermocouple sensor 112 to extend out of the coupling connector 110. It should be noted that the thermocouple and coupling connector configuration shown in FIGS. 1A-1C is an example and other configurations are contemplated. For instance, the coupling connector 110 may be made of a thermally conductive material which allows the thermocouple sensor 112 to accurately read the temperature without being exposed. As shown in FIGS. 1B and 1C, the thermocouple sensor 112 is positioned within and co-axial with the helical-shaped electrode 108. In an embodiment, the thermocouple 112 is not co-axial with the electrode 108, but located outside and adjacent to the electrode 108. In an embodiment, the thermocouple 112 is coupled to a separate wire that is insertable through the lumen of the catheter sleeve 102 and can be positioned at another location in the heart.

As shown in the embodiments in FIGS. 1B and 1C, the corkscrew shaped electrode 108 is mounted to the coupling connector 110 and is made of a conductive material which emits RF signals to heat and treat infarct tissue when the electrode 108 energized by a power source. A power wire 114 is connected to the RF generator device 150 and provides power to the electrode 108 as well as the thermocouple sensor 114. In an embodiment, separate power wires or power supplies energize the electrode 108 and the thermocouple sensor 114 as well as any other components associated with the tissue repair device 100. The electrode 108 has a length dimension of 1-5 millimeters, although other length dimensions are contemplated. The outer diameter of the electrode 108 is approximately 2 mm, whereas the inner diameter is approximately 0.5 mm. However, it is contemplated that other suitable inner and outer diameter dimensions are contemplated.

In an embodiment, the tissue insertion component 106 of the repair device 100 is configured to rotate about axis A to allow the electrode 108 to be inserted into and removed from the affected infarct tissue. When the electrode 108 initially comes into contact with the tissue, rotation of the electrode 108 about axis A will cause the electrode 108 to undergo a screw like motion into the tissue, thereby inserting itself therein. This is at least partially due to the helical configuration of the electrode 108 as well as the sharp tip of the electrode 108 as shown in FIGS. 1B and 1C. The helical or cork-screw shaped electrode 108 is advantageous considering that inserting and removing the electrode 108 into the tissue in a screw-like fashion is significantly easier for the physician than directly pushing a needle-like electrode into the tissue. In addition, the track created by the helical shape electrode 108 preserves the tissue and minimizes damage to the walls of the tissue and tissue surfaces when inserting and removing the electrode 108. The helical shaped electrode 108 provides additional advantages in that the electrode 108 is able to heat and treat a larger surface area when energized. In addition, the flux generated around the conductive rings of electrode 108 is a substantially spherical shape to treat tissue located entirely or almost entirely around the proximity of the electrode 108. It should be noted that other designs of the electrode 108 are contemplated without digressing from the inventive concepts described herein.

In an embodiment, the flexible cable 104 is rotated manually at the distal end by the user to screw the electrode 108 into and from the tissue. The user may rotate the flexible cable 104 itself or may rotate the flexible cable by using a handle 124 (FIG. 1A) located anywhere along the length of the flexible cable 104. The corkscrew electrode 108 may be screwed into and out from the tissue by rotating the rotational stability wire 116 (FIG. 1C) within the flexible cable 104. The rotational stability wire 116 is made of Nitinol in an embodiment, although other types of materials are contemplated. However, considering that the end of the cable 104 may be rigidly connected to the RF generating device 150 and/or other devices, rotation of the cable 104 after a few turns may twist the cable 104 and make it difficult to manipulate or even damage the device. Accordingly, a freely rotatable coupling device, discussed below, may be incorporated in an embodiment. It should be noted that a combination of the rotating devices described herein may be incorporated into one tissue repair device.

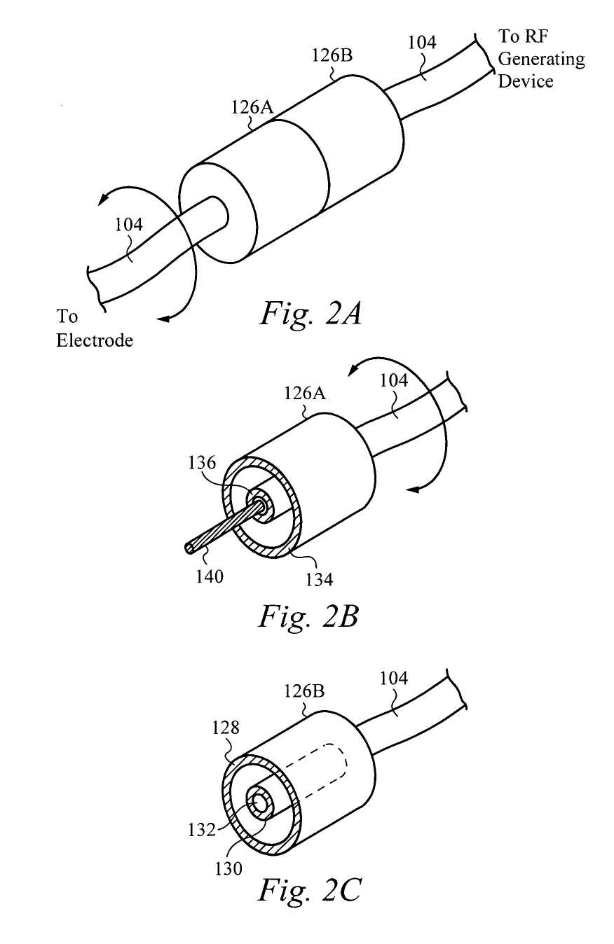

FIGS. 2A-2C illustrate diagrams of the rotatable coupling device in accordance with an embodiment. In an embodiment, the rotatable coupling device 126 includes two portions 126A and 126B coupled to one another. As shown FIG. 2A, an end of the base cable 104′ is connected to the stationary RF generating device 150, whereas the opposite end is connected to the portion 126B and supplies power thereto. In addition, an end of the flexible cable 104 is coupled to the rotatable coupling device 126A, whereby the opposite end of the cable 104 is coupled to the electrode 108. The rotatable coupling device 126 may be located anywhere along the length of the cable 104, although it is preferred that the device 126 remain proximal to the RF generating device 150 as well as outside of the catheter sleeve 102 and the patient's body.

The first portion 126A is shown in FIG. 2B in accordance with an embodiment. In the embodiment shown in FIG. 2B, the first portion 126A has an outer ring 134 and an inner ring 136, whereby the inner ring 136 is fixedly attached to the flexible cable 104. As shown in FIG. 2A, a conductive coupling protrusion 140 is attached to the flexible cable 104 and extends from the inner ring 136, whereby the coupling protrusion 140 is coupled to the power wire 114 which supplies electrical power to the tissue insertion device 106. In an embodiment, the inner ring 136 is able to rotate with respect to the outer ring 134, whereby the outer ring 134 remains stationary. This configuration allows the flex cable 104 to freely rotate along with the inner ring 136 without causing any torque to be applied to the outer ring 134. In addition, the flexible cable 104 will be able to freely rotate with respect to the base cable 104′ and remain in electrical contact therewith to easily screw the electrode 108 into the affected area without twisting the base cable 104′. In an embodiment, ball bearings are located between the outer ring 134 and the inner ring 136 to allow free rotation therebetween. However, it is contemplated that any appropriate design may be used to allow free rotation therebetween.

The second portion 126B is shown in FIG. 2C in accordance with an embodiment. The second portion 126B includes an outer ring 128 and an inner ring 132. The inner ring 126B includes a center aperture 132 which receives the coupling protrusion 140 from the first portion 126A. In an embodiment, the center aperture has an inner surface which is conductive and passes electrical signals from the cable 104′ and RF generating device 150 to the coupling protrusion 140. Thus, an electrical connection between the RF generating device 150 and the tissue repair device 106 is able to be established when the coupling protrusion 140 is received in the receiving aperture 136.

In an embodiment, the first and second portions are fixedly attached to one another as an integrated component, as shown in FIG. 2A. In an embodiment, the first and second portions are separate components which are removably coupled to one another. In that embodiment, the coupling protrusion 140 and receiving aperture 132 may be made of a magnetic material having opposite polarity, whereby the protrusion 140 may be removably coupled to the aperture 132. The magnetic coupling protrusion 140 would be conductive and electrically connected to the aperture 132 when coupled thereto.

In an embodiment, the rotatable coupling device 126 is configured to measure and track the rotational movement of the flexible cable 104 during the procedure. Any appropriate type of sensor may be incorporated into the coupling device 126, whereby the sensor would track the number of rotations of the cable 104 (and thus the electrode 108) and send signals to a processor of a feedback system. The feedback system may be a computer program run on a host computer which is configured to store, analyze and display the measured information to the user to keep track of how many revolutions are performed and/or needed to effective screw the electrode 108 to a desired depth in the heart tissue. In an embodiment that the user utilizes both the handle 124 as well as the coupling device 126, sensors may be incorporated in the handle 124 and coupling device 126 to measure and display data of the relative rotations of each. In another embodiment, an indicator is located directly on the handle 124 and/or coupling device 126 to indicate the number of rotations undergone during the procedure.

The RF generating device 150 provides modulated power to the resistive corkscrew electrode 108 to emit an RF signal at a selected frequency and magnitude. The frequency is in the range of 10 MHz to 1000 MHz. The RF signal emitted from the electrode 108 is converted into heat by the affected tissue, whereby heating of the affected tissue to a desired temperature causes reduction of the surface area in the affected infarct tissue without ablating the affected tissue or damaging the healthy tissue surrounding the affected area. The affected tissue is heated by the electrode 108 under dynamic conditions having variable strain created by the heart muscle itself which may aid in improving the reduction of the affected tissue's size and/or thickness.

The RF generating device 150 applies between 1 W to 40 W to the electrode 108 to effectively heat the affected tissue between 40° C. and 75° C. for optimum reduction of the affected tissue. In an embodiment, the RF generating device 150 has a single channel and delivers the power to the electrode 108 continuously. In an embodiment, the RF energy emitted at the electrode 108 may be multiplexed by applying the energy in different waveform patterns (e.g. sinusoidal wave, sawtooth wave, square wave) over time as appropriate. In an embodiment, the affected tissue is continuously heated by the electrode for a desired amount of time. It should be noted that other power levels, desired temperatures, desired time periods, and/or energy patterns are contemplated based on the type of affected tissue, materials used in the device 100, frequencies and other factors.

A feedback system may be employed to the electrode 108 for detecting appropriate feedback variables during the treatment procedure. In an embodiment, the thermocouple sensor 112 senses the temperature of the infarct tissue during treatment and sends those signals to a processor which provide feedback to allow the system 100 to automatically or manually modulate the power supplied by the RF generator 50 to the electrode 108. The thermocouple 112 senses the temperature of the tissue through its tip (FIG. 1B) or through the conductive material of the coupling connector. As stated above, optimum reduction of the infarct tissue is achieved when the tissue is heated between 40° C. and 75° C. Accordingly, the thermocouple sensor 112 measures the temperature of the tissue as it is treated and outputs a signal associated with the measured temperature to processor 122 integral or separate from the RF generating device 150. It should be noted that the sensor may measure other variables (e.g. pressure) instead of or in addition to temperature.

In an embodiment, the processor 122 compares the measured temperature with a desired or preprogrammed temperature and accordingly informs the user or automatically causes the RF generating device 150 to alter the power supplied to the electrode 108. As the thermocouple 112 measures the affected tissue reaching the desired temperature, the processor 122 continuously receives the information from the thermocouple 112 and provides signals to the RF generating device 150 to increase, decrease, modulate, reinitiate or terminate power to the electrode 108. In an example, the system 100 is configured such that the RF generating device 150 automatically terminates power supplied to the electrode 108 upon the thermocouple 112 indicating the affected tissue has reached the desired temperature. In an example, the system 100 automatically produces an audible sound and/or video display indicating that the affected tissue has reached the desired temperature. In an embodiment, the affected tissue is heated continuously by the electrode for a desired amount of time before or after the desired temperature has been reached. In an embodiment, the affected tissue is heated continuously by the electrode after the desired temperature has been reached until the infarct tissue shrinks or has been reduced a maximum allowable amount for a treatment. It is contemplated that a computer display coupled to the processor and is configured to provide graphical data of the sensed temperature of the tissue and/or a graphical simulation of the tissue treatment process. In an embodiment, a display may be used to show an actual video image of the electrode within the heart tissue in real time, whereby the surgeon is able to see the actual reduction of the infarct tissue as it is heated by the electrode. This provides visual feedback to the surgeon to alter or terminate the modulated power to the electrode if the infarct tissue is no longer shrinking.

In an embodiment, the thermocouple sensor 112 acts as a tissue depth limiting device. As shown in FIG. 1C, the thermocouple sensor 112 is positioned within the helical electrode 108, whereby the tip of the sensor 112 is 2-3 mm from the tip of the electrode 108 in an embodiment, although other dimensions are contemplated. The thermocouple 112 is configured to come into contact with the tissue as the electrode 108 is inserted and effectively blocks or prevents the electrode 108 from going any further into the tissue. In an embodiment, the thermocouple 112 is configured to measure and provide temperature information as the electrode 108 is being inserted into the tissue, whereby a sudden increase in the temperature measured by the thermocouple 112 will notify the user that the sensor 112 has come into contact with the tissue itself. Thus, the user will be able to tell that the electrode 108 has been inserted to a maximum depth into the tissue. This may be useful in the embodiment in FIG. 1B where the sensor 112 is co-axial with the electrode 108 and thus conveniently serves as the tissue depth limiting device. In an embodiment, the thermocouple 112 itself is resistive and emits RF signals when applied with the modulated power, whereby the thermocouple 112 treats the affected tissue.

The configuration of the electrode 108 allows flexibility in treating the affected tissue irrespective of the location of the affected tissue in the heart wall. In addition, the ability for the electrode 108 to be inserted directly into the tissue provides information as to the depth of the infarct tissue while potentially protecting one ore both surfaces of the heart tissue. For example, the electrode 108 may be directly inserted into the infarct tissue to treat the affected tissue. The electrode 108 may be inserted into healthy heart tissue to treat and repair infarct tissue located adjacent to or below healthy tissue, without heating the healthy tissue. In the case of the infarct tissue being located below the healthy tissue, infarct tissue located proximal to or on the outer wall of the heart may be effectively treated even though the electrode is inserted from the heart's inner wall. In an embodiment, the electrode is inserted into healthy tissue which is adjacent to the infarct tissue to effectively treat and repair the infarct tissue without heating the healthy tissue. In an embodiment, the electrode may be inserted into healthy tissue located between two areas of infarct tissue to treat both areas simultaneously or individually without heating the healthy tissue. In contrast, the electrode may heat an affected tissue layer located between two healthy tissue layers without heating the healthy layers. In a scenario, the electrode may be heated using one or more heating patterns to allow a controlled depth heating of affected tissue areas interspersed within healthy tissue. Upon treating the infarct tissue, the electrode may be easily removed from the heart tissue and reinserted into another location in the heart to treat another infarct tissue or another area or portion of the previously treated infarct tissue.

In an embodiment, the tissue insertion device 106 has a mono-polar configuration, whereby a ground potential 118 is placed at a location not within the immediate proximity of the electrode 108. The mono-polar configuration allows RF signals emitted by the electrode 108 to spread over a larger area of the affected tissue considering the receiving ground potential is not in immediate proximity but a distance away from the electrode 108. The ground potential 118 can be a conductive grounded receiving wire similar in size to the electrode 108 which is placed on or near the patient's skin and may or may not be connected to the RF generating device 150. In an embodiment, the receiving electrode is placed on the patient's back during the procedure. In an embodiment, the receiving wire is placed in proximity to the location of the electrode 108 within the patient's heart to allow somewhat focused transmission of the RF signals to the receiving wire. In an embodiment, a polarity opposite to that emitted by the electrode 108 is applied to the receiving wire, whereby the opposite polarity can be generated by the RF generating device 150. In an embodiment, the device has a bi-polar configuration, one or more embodiments of which is described below.

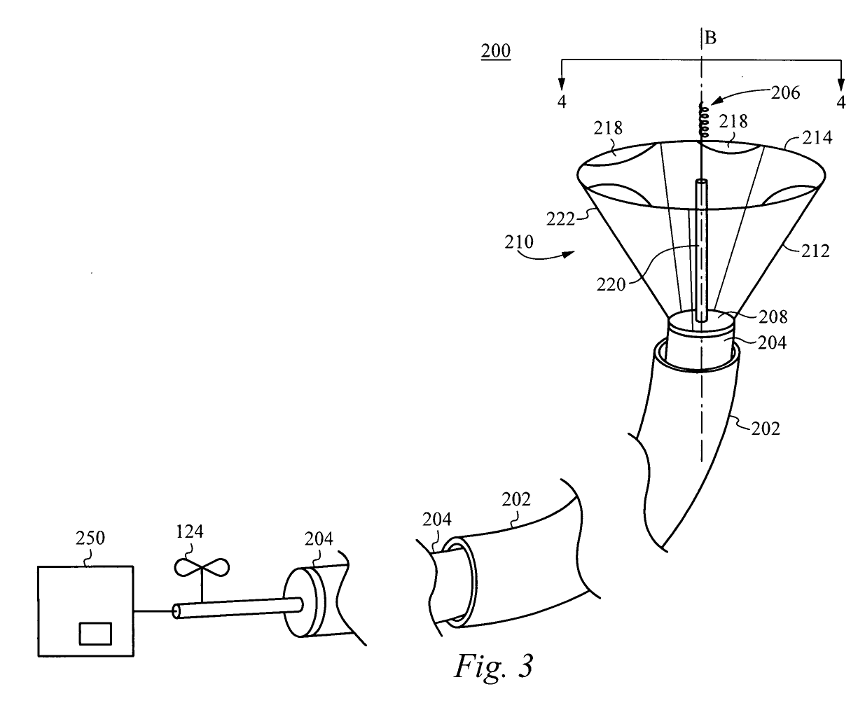

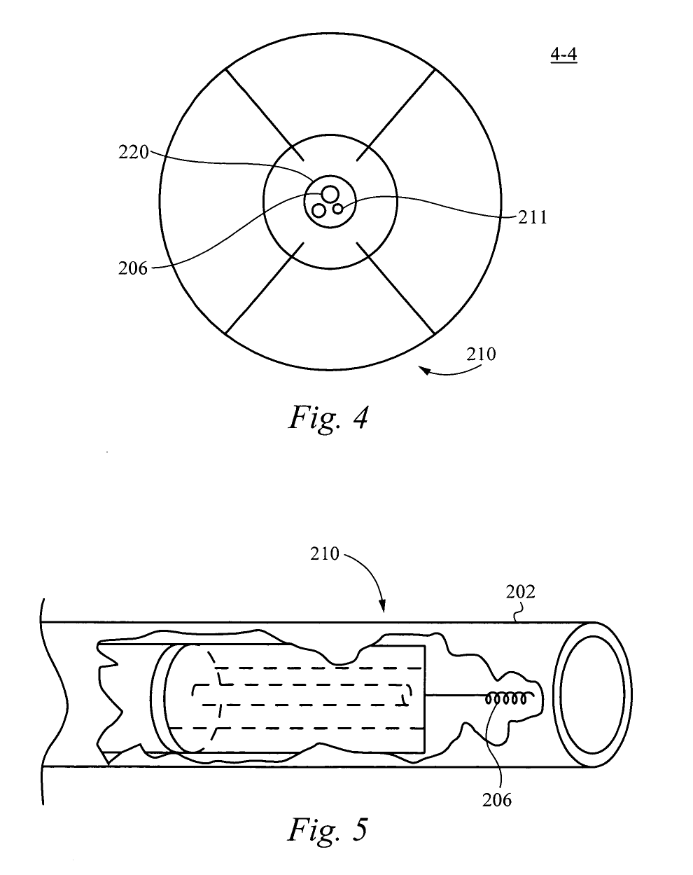

FIG. 3 illustrates a schematic of a tissue repair device in accordance with an embodiment. In an embodiment, the tissue repair device 200 includes a catheter sleeve 202 configured to receive a flexible cable 204 of the tissue repair device. In the embodiment shown in FIG. 3, the tissue insertion device 204 includes a collapsible tissue repair component 210 which comprises a ring 208 coupled to a distal end of the flexible cable 204. The tissue insertion device 200 includes a plurality of struts 212 with an end coupled to the ring 208. The struts 212 are flexible and spring-like to be capable of flexing toward and away from a center of the ring 40. In an embodiment shown in FIG. 3, an opposite end, hereinafter distal end, of the struts 212 are coupled to a flexible wire 214 in a circular configuration to limit the outward motion of the distal ends of the struts 212.

In the embodiment shown in FIG. 3, a center electrode 216 is located along an axis of the ring 208, and a plurality of outside electrodes 218 are mounted to the wire 214. The center electrode 220 and outside electrodes 218 are electrically connected to the RF generating device 250 that is located outside the patient's body. As opposed to the mono-polar configuration described above, the center electrode 220 emits the RF signals whereas the outside electrodes 218 receive the signals to effectively spread the RF signals through the tissue between the center electrode 220 and outside electrodes 218. In an embodiment, the outside electrodes 218 are grounded. Alternatively, the outside electrodes 218 have an opposite polarity to that of the center electrode 220. It is contemplated, however, that the any of the embodiments described herein may utilize either the mono-polar or bi-polar configuration.

In an embodiment, Mylar is used to form a bag-like structure 222 which is located around the collapsible tissue repair component 210 to completely enclose the struts 212, wire 214 and electrodes 218, 220, whereby the proximal end of the Mylar sheet 222 is connected to the ring 208. In an embodiment, the electrodes 218, 220 may be an integral part of the Mylar sheet 222. In an embodiment, the electrodes 218, 220 may be printed in electrically-conductive ink on the Mylar 222. In an embodiment, the Mylar sheet 222 itself can act as a restraint on the struts 212, thereby obviating the need for the wire 214. It should be noted that Mylar is an example material and other appropriate materials are contemplated for use with the device described herein.

As shown in FIG. 3, a thin, flexible rod 224 extends through a lumen in the flexible 204 as well as through a lumen in the center electrode 220. As with the embodiment in FIGS. 1A-1C, a corkscrew-shaped electrode 206 is located at the distal end of the wire 224, and a handle 226 is configured at the proximal end of the wire 224 so that a user can rotate the handle 226 to cause the corkscrew-shaped connector 74 to rotate, as stated above.

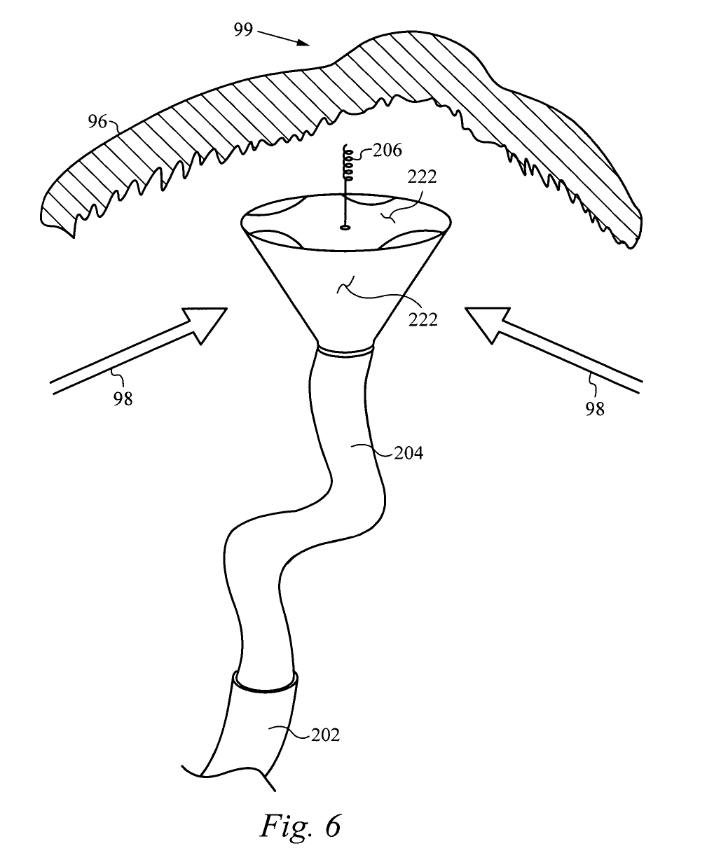

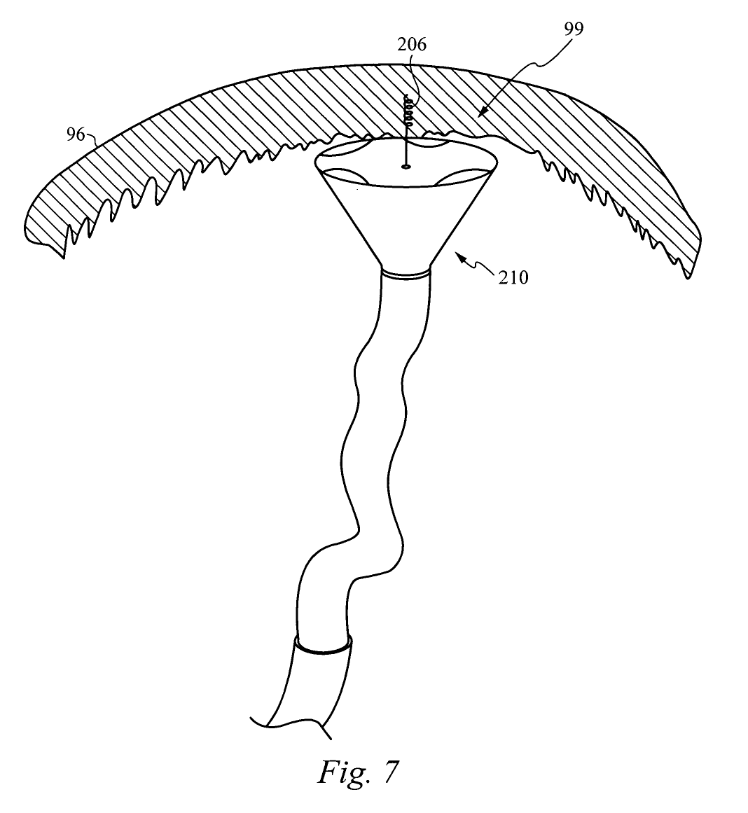

FIGS. 6 and 7 illustrate a self positioning collapsible tissue repair component in use to treat affected tissue in the heart in accordance with an embodiment. For the collapsible tissue repair component, the device itself may be used to locate the infarcted portion 99. In some cases, the infarcted portion 99 is somewhat thinner and non-contractile, unlike than the adjacent, healthy portion of the heart. Consequently, when the heart muscles contract, the infarcted portion 99 bulges outward from its normal configuration, as indicated in FIG. 6, or simply does not add to the movement of blood out of the heart due to its non-contractile characteristics. When this occurs, there tends to be blood flow toward the bulge or dyskinetic area, or toward the non-contracting area called the akinetic area as suggested by arrows 98. Accordingly, the collapsible tissue repair component 210 may self position itself by acting like a sail and being carried toward the dyskinetic or akinetic area of the heart by the blood flow. In this embodiment to facilitate the self-positioning feature, at least the flexible cable 41 and in some cases, both the flexible cable 41 and the catheter 31, should be considered instead of a conventional catheter. Specifically, a conventional catheter is relatively rigid and can include structures to permit a physician to manipulate the distal end of the catheter from a location external to the patient. Such a catheter can be called a “steerable” catheter. In contrast, at least the flexible cable 204 and in some cases, both the flexible cable 204 and the catheter 202 should be flexible or floppy to allow the blood flow to move the collapsible tissue repair component 210 toward the infracted tissue. For this reason, the flexible cable 204 is shown in FIGS. 6 and 7 as somewhat limp, and the catheter 202 can be understood to be a flexible tube, without the components often found in a conventional steerable catheter, to permit the user to manipulate the distal end of the catheter from a location external to the patient. Similarly, the flexible tube 204 may not include components which permit a user to manipulate the distal end of the flexible tube from a location external to the patient.

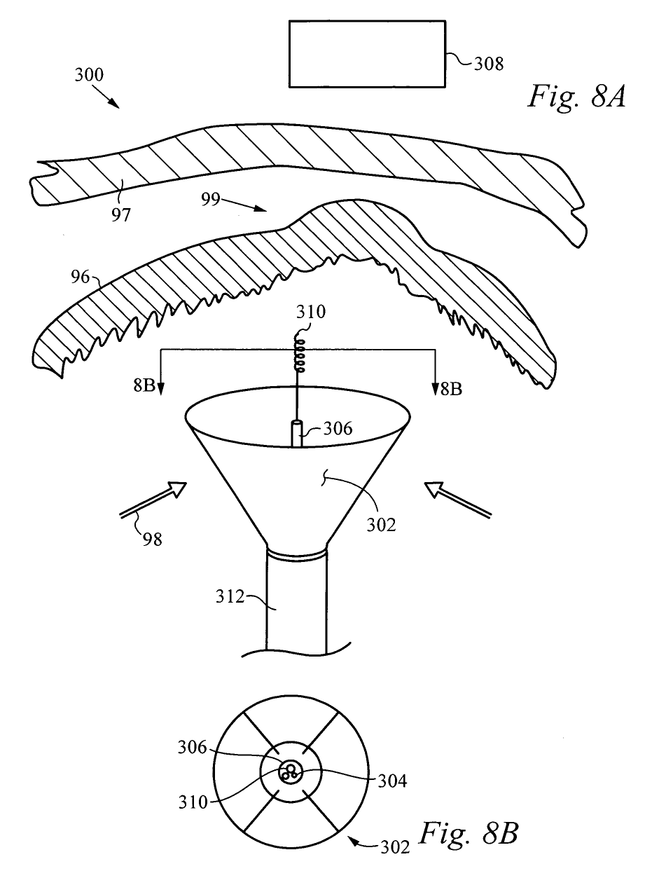

FIGS. 8A and 8B illustrate another electrode locating system in accordance with an embodiment. The embodiment in FIGS. 8A and 8B is directed to an electrode locating system 300 which comprises a collapsible tissue repair component 302 having an ultrasonic crystal 302 mounted at the distal end of the center electrode 306 (FIG. 8B). The embodiment in FIGS. 8A and 8B further comprises a locating device 308 having an ultrasonic crystal array which is located outside the patient 97 and which allows a user to determine the location of the ultrasonic crystal 304 and electrode 310 inside the patient. The embodiment in FIGS. 8A and 8B further includes a steerable catheter 312, and the repair device 302 is mounted to the distal end of the steerable catheter 312.

In operation for the embodiment in FIGS. 8A and 8B, the repair device 310 is introduced into the patient's heart. The user then uses the locating device 308 to monitor the location of the ultrasonic crystal 304 and thus the repair device 302, whereby the user manipulates the steerable catheter 312 to position the electrode 310 to be in contact with the infarct tissue 99. The user then operates the device as mentioned by one or more embodiments described herein.

It should be understood that other types of monitoring and locating systems could be used by a physician to monitor the location of a tissue repair device to properly insert the electrode into the affected infarct tissue. In an embodiment, an electrocardiogram (ECG/EKG) of the heart tissue may be used to monitor the position of the electrode and tissue repair device within the heart in real time. It would be preferred that the electrode or other portion of the tissue insertion device is made of a material which is able to be easily displayed in an ECG/EKG. Particulars of the ECG/EKG are well known in the art and are not described herein.

In an embodiment, magnetic resonance imaging (MRI) may be utilized to monitor the position of the tissue repair device within the heart in real time, whereby magnetic fields are used to orient and move the tissue repair device to the desired affected area. In an embodiment, the electrode of the tissue repair device may emit magnetic fields, instead of RF energy, to heat and thereby heal the affected infarct tissue.

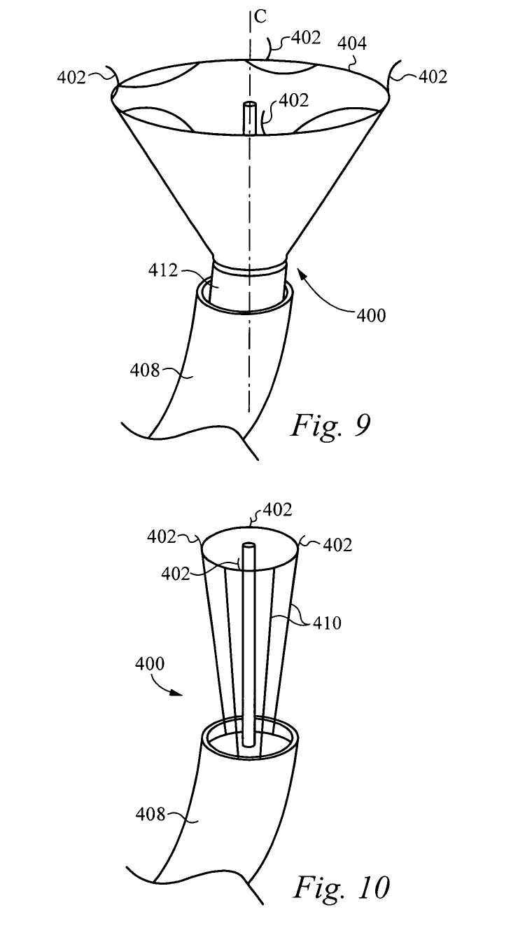

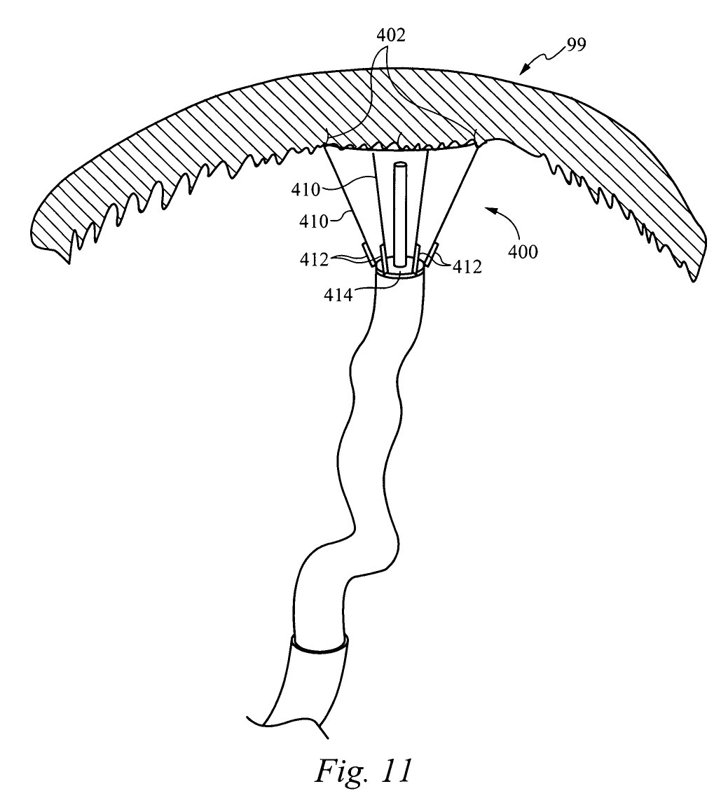

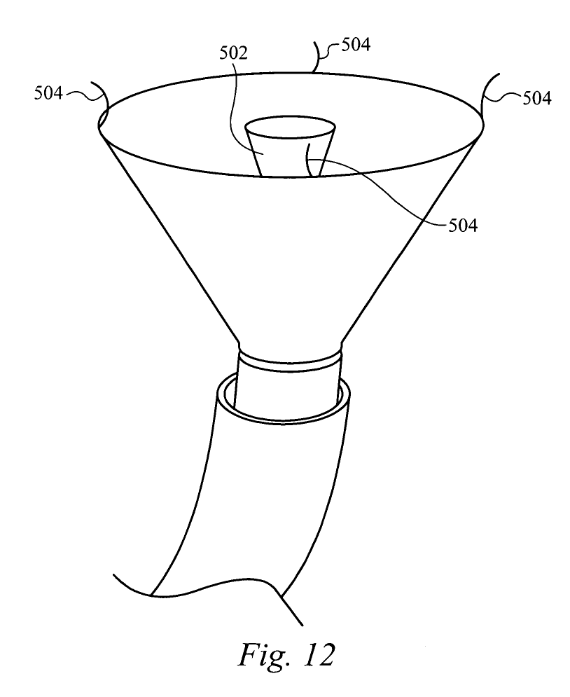

FIGS. 9-11 illustrate another embodiment of the tissue repair device. In particular, the tissue repair device 400 is similar to tissue repair device described above with the addition of a plurality of hooks 402 are disposed around the periphery of the wire 404. It should be noted that although the corkscrew shaped electrode is not shown in FIGS. 9 and 10, the tissue repair device 400 may alternatively include the corkscrew shaped electrode extending from the catheter electrode 406. In an embodiment, the hooks 402 are concave with their middle portions being closer to the central axis C than their top and bottom portions. In operation, the tissue repair device is pushed through the catheter 408 until it nears the distal end of the catheter. At this point the distal end of the catheter 408 can be positioned in contact with or adjacent to the infarct. Then, as the tissue repair device 400 exits the distal end of the catheter 408 (FIG. 10 not showing the Mylar coating), the struts 410 begin to move away from their collapsed orientation and the hooks 402 engage the infarct as shown in FIG. 11. When the operation has been completed, the hooks 402 are released from the infarct tissue 99 by sliding the distal end of the catheter over the struts 410.

In an embodiment, as shown in FIG. 11, the tissue repair device 400 includes strain gauges 412 connected to the struts 410 and the ring 414 to measure flexion of the struts 410 relative to the ring 414. Specifically, when the hooks 402 are inserted into the infarcted portion, the strain measured by the strain gauges 412 is recorded. The strain gauges 412 then send signals associated with the sensed data to a processor outside the patient. The processor is then able to record and display the measurement data of the extent to which the infarcted portion 99 has been treated. The physician is then able to accurately assess from this data the amount of shrinkage the infracted tissue has undergone during the treatment in real time. When the measured strain stops changing, the physical is notified that the infarct portion is completely treated and will not shrink any further. At this time, treatment is completed, and the repair device 400 is removed from the infarct tissue.

FIG. 12 illustrates an embodiment of the treatment device. According to the embodiment shown in FIG. 12, the tissue repair device 500 does not include a center electrode or outside electrodes, but rather, an infrared light source 502 which is connected to a controllable power supply (not shown). The infrared light source 502, like the other embodiments, is used to heat the infarct portion of the patient to repair the tissue.

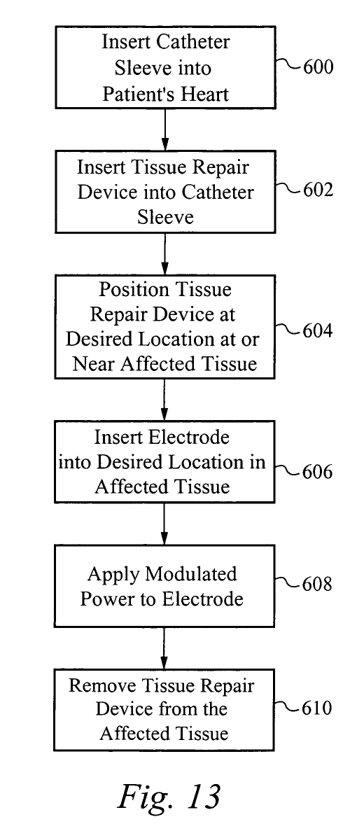

FIG. 13 is a flow chart illustrating the method of utilizing the tissue repair device shown and described herein. It should be noted that the method described herein may applied to any or all of the embodiments described, unless otherwise specified. As shown in FIG. 12, a physician initially introduces the catheter sleeve into a patient so that the distal end of the catheter is positioned in the interior of the patient's heart (600). The physician then inserts the tissue repair device into the proximal end of the catheter sleeve (602) and pushes the repair device out to or near the distal opening of the catheter sleeve. At substantially the same time, the physician utilizes a locating device or method described above to accurately position the tissue insertion portion of the repair device to be close to the affected tissue (604). Positioning is done by manipulating the flexible cable and catheter sleeve or by utilizing a steerable catheter, as described above. In the collapsible device embodiment, as the physician continues to push the flexible cable through the catheter sleeve, the collapsible repair device exits the distal end of the catheter sleeve and expands to the deployed orientation as shown in FIG. 3. For the non-collapsible device embodiment shown in FIG. 1, the physician simply pushes the corkscrew electrode out of the catheter sleeve.

Once it is determined that the electrode is at the desired position with respect to the infarct tissue, the physician rotates the flexible cable itself or a handle to rotate the corkscrew electrode to insert and engage the electrode into the infarct tissue (606). Alternatively, the cable may be rotated automatically. Modulated power is then applied to the electrode, whereby the electrode emits RF signals directly into the infarct tissue (608). A temperature sensor of the repair device may be used to sense the temperature of the infarct tissue. As stated above, the modulated power level is 1 W-40 W and the frequency of the signals is in the range of 10 megahertz to 1000 megahertz, to heat the scar tissue to a temperature sufficient to reduce the surface area of the scar without ablating the scar tissue or damaging the healthy tissue surrounding the infarct tissue. The scar tissue is heated in the range of about 40 degrees Celsius to about 75 degrees Celsius.

Once the infarct tissue has reached a desired temperature for a desired period of time, the treatment is completed. The period of time is between 1 and 2 minutes in an embodiment, although other periods of time are contemplated based on a variety of factors including, but not limited to, wattage, frequency, and size of electrode. Thereafter, the flexible cable is rotated the opposite direction than before to remove the electrode from the infarct tissue (610). Upon treating the infarct tissue, the electrode may be easily removed from the heart tissue and reinserted into another location in the heart to treat another infarct tissue or another area or portion of the previously treated infarct tissue. The tissue repair device is then removed from the catheter sleeve, wherein the catheter sleeve is then removed from the patient.

While embodiments and applications of this tissue repair device have been shown and described, it would be apparent to those skilled in the art having the benefit of this disclosure that many more modifications than mentioned above are possible without departing from the inventive concepts herein.

What is claimed is:

1. A system for treating an affected portion in a heart, comprising:
a catheter having a first end and a second end;
an electrode coupled to the first end, wherein the electrode is adapted to be rotatably inserted into heart tissue;
a power source coupled to the second end and configured to energize the electrode, wherein the electrode emits a radio frequency (RF) signal upon being energized to heat the affected portion to a desired temperature; and
a temperature feedback control coupled to the electrode and the power source, wherein the power supply is configured to adjust the emitted RF signal based on a measured temperature of the affected portion.

2. The device of claim 1, further comprising a rotatable member coupled to the catheter and positioned between the electrode and the power source, the rotatable member configured to allow the electrode to freely rotate with respect to power source.

3. The device of claim 2, wherein the rotatable member is configured to measure a number of rotations of the electrode with respect to the power supply when the electrode is inserted into the heart tissue.

4. The device of claim 1, wherein the electrode further comprises a helical configuration configured to allow the electrode to be rotatably inserted into the heart tissue.

5. The device of claim 1, wherein the temperature feedback control further comprises a temperature sensor.

6. The device of claim 5, wherein the temperature sensor is configured to provide depth information of the electrode being inserted into the heart tissue.

7. The device of claim 4, wherein the temperature feedback control further comprises a temperature sensor positioned within and coaxial with the helical electrode.

8. The device of claim 1, wherein the desired temperature in the range of about 40 degrees Celsius to about 75 degrees Celsius.

9. The device of claim 1, wherein the electrode has a mono-polar configuration.

10. The device of claim 1, wherein the electrode has a bi-polar configuration.

11. The device of claim 1, wherein the electrode is configured to be energized for a predetermined amount of time.

12. The device of claim 1, wherein the electrode is configured to be energized for a predetermined amount of time after the desired temperature has been reached.

13. The device of claim 1, wherein the electrode is configured to be energized after the desired temperature has been reached until the affected portion is reduced a maximum allowable amount.

14. The device of claim 1, wherein the electrode is configured to be viewed on a display.

15. A device for treating an affected portion in a heart, comprising:
a catheter;
an electrode coupled to an end of the catheter and configured to be inserted into heart tissue, the electrode configured to emit a radio frequency (RF) signal upon being energized to heat the affected portion to a desired temperature; and
a temperature sensor coupled to the catheter, the temperature sensor configured to measure a temperature of the affected portion.

16. The device of claim 15, further comprising a rotatable member coupled to the catheter, the rotatable member configured to allow the electrode to freely rotate with respect to a ground.

17. The device of claim 16, wherein the rotatable member is configured to measure a number of rotations of the electrode with respect to ground when the electrode is inserted into the heart tissue.

18. The device of claim 15, wherein the electrode further comprises a helical configuration configured to allow the electrode to be rotatably inserted into the heart tissue.

19. The device of claim 15, further comprising a power source coupled to the electrode, wherein the power source energizes the electrode.

20. The device of claim 15, wherein the temperature sensor is configured to provide depth information of the electrode being inserted into the heart tissue.

21. The device of claim 15, wherein the temperature sensor is positioned within and coaxial with the helical electrode.

22. The device of claim 15, wherein the desired temperature in the range of about 40 degrees Celsius to about 75 degrees Celsius.

23. The device of claim 15, wherein the electrode has a mono-polar configuration.

24. The device of claim 15, wherein the electrode has a bi-polar configuration.

25. The device of claim 15, wherein the electrode is configured to be energized for a predetermined amount of time.

26. The device of claim 15, wherein the electrode is configured to be energized for a predetermined amount of time after the desired temperature has been reached.

27. The device of claim 15, wherein the electrode is configured to be energized after the desired temperature has been reached until the affected portion is reduced a maximum allowable amount.

28. A device for treating an affected portion in a heart, comprising:
an electrode configured to be inserted into heart tissue at least proximal to the affected portion;
means for energizing the electrode to emit a radio frequency (RF) signal to heat the affected portion; and
means for measuring a temperature of the affected portion, wherein the means for energizing the electrode alters power supplied to the electrode based on the measured temperature.
